(19) United States
(12) Patent Application Publication
(10) Pub. No.: US 2015/0147765 A1
(43) Pub. Date: May 28, 2015

(54) SEROLOGICAL MARKERS FOR CANCER DIAGNOSIS USING BLOOD SAMPLE

(71) Applicant: Korea Basic Science Institute, Daejeon (KR)

(72) Inventors: Yeong Hee Ahn, Chungcheongbuk-do (KR); Jong Shin Yoo, Seoul (KR); Eun Sun Ji, Daejeon (KR)

(21) Appl. No.: 14/303,939

(22) Filed: Jun. 13, 2014

(30) Foreign Application Priority Data
Nov. 28, 2013 (KR) 10-2013-0145983
May 30, 2014 (KR) 10-2014-0066110

(51) Int. Cl. G01N 33/68

(52) U.S. Cl. CPC G01N 33/6818
USPC 435/7.92; 436/89

(57) ABSTRACT

The present invention relates to a method for diagnosing cancer using information on the aberrant glycosylation of a glycoprotein involved in cancer progress. More particularly, the present invention relates to a cancer diagnosis peptide marker which is screened by the steps of: separating a glycoprotein aberrantly glycosylated according to the occurrence and progress of cancer from the blood of a lung cancer patient by using lectin; and selecting a marker peptide produced by the hydrolysis of the glycoprotein isolated by lectin. The marker peptide can be effectively used as a cancer diagnosis marker and for the diagnosis of cancer.

This patent application claims the benefit of priority from Korean Patent Application No. KR10-2013-0145983 filed Nov. 28, 2013 and Korean Patent Application No. KR10-2014-0066110, filed May 30, 2014, the contents of each of which are herein incorporated by reference in their entirety.

### FIELD OF THE INVENTION

The present invention relates to a method for diagnosing cancer which comprises the steps of selecting a polypeptide originated from a specific glycoprotein existing in cancer patient blood and analyzing the marker polypeptide qualitatively and quantitatively.

### BACKGROUND

Glycosylation is one of the most representative protein modification processes. In many glycoproteins secreted from cancer cells or found on the surface of cell membrane, aberrant glycosylation is observed over the occurrence and progress of cancer. It has been reported that many diseases are related to abnormal actions of glycosyltransferase and glycolytic enzyme mediated by abnormal signal transduction of cancer genes (Orntoft, T. F.; Vestergaard, E. M. Clinical aspects of altered glycosylation of glycoproteins in cancer. Electrophoresis 1999,20:362-71).

Such aberrant changes in the glycosylation pattern as the increase of the size of n-linked sugar chain and the number of side chain, the increase of sialyation and fucosylation, and the formation of polylactosamine causing changes in the size of sugar chain are observed over the progress of cancer. So, the glycoprotein can be used efficiently as a cancer marker that can detect the occurrence and progress of cancer by analyzing the above changes related to the glycoprotein. Particularly, the abnormal increase of fucosylation in cancer cells provides a possibility to distinguish the proteins in cancer cells from the proteins in normal cells. Therefore, the aberrantly glycosylated glycoprotein can be developed as a cancer marker for diagnosing cancer. That is, a method to analyze abnormally fucosylated protein glycoforms other than the glycoprotein itself is required. Glycoproteins containing information on cancer are secreted in extracellular media or are shed from cell membrane once they accomplish their roles. Thus, various cancer cell culture media, cancer tissue lysis, and cancer patient blood are all good materials for the detection of cancer markers which are glycoproteins containing information on cancer, etc.

Analysis of difference in protein glycosylation between the normal group and the cancer patient group provides an important clue to distinguish cancer patients from normal people. Therefore, it has been attempted to develop many methods to analyze the difference. To investigate the differences in glycosylation, glycoproteins or glycopeptides are separated and concentrated by using selectivity of lectin to glycostructure of a glycoprotein. According to the structure of glycochain, ConA (Concanavalin A), WGA (Wheat germ agglutinin), Jacalin, SNA (Sambucus nigra agglutinin), AAL (Aleuria aurantia lectin), L-PHA (Phytohemagglutinin-L), PNA (Peanut agglutinin), LCA (Lens culimaris agglutinin-A), ABA (Agaricus biflorus agglutinin), DBA (Dolichos biflorus agglutinin), DSA (Datura stramonium agglutinin), ECA (Erythrina cristagalli agglutinin), SBA (Soybean agglutinin), SSA (Sambucus sieboldiana agglutinin), UEA (Ulex europaeus agglutinin), VVL (Vicia villosa lectin), BPL (Bauhinia purpurea lectin), or multilectin which is prepared by mixing some of the above lectinc is used (Yang, Z. et al., J. Chromatogr, A, 2004, 1053, 79-88., Wang, Y. et al., Glycobiology, 2006, 16, 514-523). This method is based on the selectivity of lectin to glycostructure of glycoprotein, so that it is advantageous in selective isolation and concentration of glycoproteins having specific glycochain structure. Particularly, this method facilitates the elimination of those proteins that do not have affinity to lectin through the process of lectin specific glycoprotein separation, suggesting that this method has the advantage of reducing complexity of test samples. The isolated/concentrated glycoproteins can be analyzed qualitatively and quantitatively by various electrochemical methods, spectrochemical methods, and particularly mass spectrometric methods.

One of the most frequently hired methods is lectin-blotting that is to analyze glycoproteins using selectivity of lectin to glycostructure of a glycoprotein. In general, this method is co-used with immunoblotting demonstrating high selectivity to a specific protein. Therefore, an antibody against an antigen glycoprotein is necessary, in other words this method cannot be performed with those proteins without matching antibodies. Another disadvantage of lectin-blotting based on the gel-separation technique is found in analysis speed and quantification reliability. To increase analysis speed and sensitivity of the conventional lectin-blotting, sandwich array using an antibody and lectin is used recently (Forrester, S. et. al., Low-volume, high-throughput sandwich immunoassays for profiling plasma proteins in mice: identification of early-stage systemic inflammation in a mouse model of intestinal cancer. Mol Oncol 2007, 1(2): 216-225). However, this method also requires reliable antibodies and it is still very difficult to obtain the antibodies against all the newly identified glycoproteins.

In the meantime, mass spectrometry is very efficient in high speed high sensitive quantitative/qualitative analysis of very complicated proteome samples. In particular, multiple reaction monitoring mass spectrometry (MRM MS) facilitates fast and reliable quantification of a polypeptide having a comparatively small mass produced from hydrolysis of a protein. Even the protein having no antibody matched can be quantitatively analyzed by this method. Precisely, MRM is a high sensitive quantitative analysis method to analyze selectively the target peptide obtained from a very complicated sample through one or more liquid chromatography to separate peptides and two times of precursor mass selection and fragment ion selection (Anderson L, et al., Mol. Cell Proteomics. 2006, 5, 573-588). It is very difficult to detect and analyze quantitatively the plasma biomarker protein existing in a small volume with a low concentration in a sample, such as plasma, where other proteins exist at high concentration. So, to detect a disease biomarker in plasma, the most dominant proteins such as albumin, lgG, IgA, transferrin, and haptoglobin have to be eliminated in order to minimize the complexity of a sample and to facilitate the analysis with the target protein alone. However, this process is not always necessary. Even though the complexity of a sample is minimized by the elimination of high concentration plasma proteins and the target peptide selectivity is increased by LC-MRM, if the amount of a target marker protein in the sample is extremely low, concentrating the marker protein by using antibody immunoaffinity or concentrating the hydrolyzed marker peptide is required to improve LOD (Limit of Detection) and LOQ (Limit of Qualification) of the cancer marker. Even so, the target marker protein or marker peptide specific antibody has to be prepared.

The present inventors tried to develop a marker for cancer diagnosis. As a result, the present inventors succeeded in the separation and concentration of aberrantly glycosylated glycoproteins generated in lung cancer patient blood by using lectin; in obtaining polypeptides thereof by hydrolyzing the said glycoproteins; and in selecting the hydrolyzed marker peptides originated from the marker (Complement factor I) proteins demonstrating cancer specific glycosylation through sequence analysis and quantitative analysis, leading to the completion of this invention by confirming that the said peptide markers can be effectively used as cancer diagnosis markers and for the method for diagnosing cancer.

### SUMMARY OF THE INVENTION

It is an object of the present invention to provide a method for diagnosing cancer by using a marker peptide that is able to detect glycoprotein specific quantitative changes generated over the occurrence and progress of cancer.

It is another object of the present invention to provide a cancer diagnosis kit and biochip using the marker peptide of the present invention.

To achieve the above objects, the present invention provides a method for diagnosing cancer comprising the following steps:

1) separating and concentrating glycoproteins from the sample obtained from a test subject;

2) preparing polypeptides by hydrolyzing the glycoproteins of step 1);

3) sequencing and/or quantitative-analyzing the polypeptides of step 2); and

4) diagnosing the test subject with high risk of cancer or with cancer if the polypeptide composed of the amino acid sequence represented by SEQ ID NO:1 or the polypeptide having the molecular weight of 1191.6 is detected from the sequencing or quantitative analysis of step 3).

The present invention also provides a method for diagnosing cancer comprising the following steps:

1) separating and concentrating glycoproteins from the sample obtained from a test subject;

2) preparing polypeptides by hydrolyzing the glycoproteins of step 1;

3) sequencing and/or quantitative-analyzing the polypeptides of step 2); and

4) diagnosing the test subject with high risk of cancer or with cancer if the polypeptide composed of the amino acid sequence selected from the group consisting of the sequences represented by SEQ ID NO:1 through SEQ ID NO:19, or the polypeptide having the molecular weight of 1191.6, 993.4, 979.6, 1113.6, 1296.7, 1093.7, 1014.6, 1195.6, 1370.8, 1369.7, 1291.7, 1354.8, 1140.6, 1026.5, 1250.6, 1115.6, 1139.6, 1097.6, or 1183.6 is detected from the sequencing or quantitative analysis of step 3).

The present invention also provides an antibody specifically binding to the polypeptide composed of the amino acid sequence represented by SEQ ID NO:1. or a kit for diagnosing cancer comprising the antibody.

The present invention also provides a kit for diagnosing cancer comprising an antibody specifically binding to each polypeptide composed of the amino acid sequence respectively represented by SEQ ID NO:1 through SEQ ID NO:19 or a combination of those antibodies.

The present invention also provides a biochip for diagnosing cancer wherein biomolecules specifically binding to the polypeptide composed of the amino acid sequence represented by SEQ ID NO:1 are integrated on the solid substrate.

The present invention provides a biochip for diagnosing cancer which is characterized by the further integration of biomolecules specifically binding to each polypeptide composed of the amino acid sequence represented by SEQ ID NO:1 through SEQ ID NO:19.

In addition, the present invention also provides a method for diagnosing cancer comprising the following steps:
1) measuring the expression level of Complement factor I from the sample obtained from a test subject;
2) selecting a subject demonstrating the increased expression level of CFI, compared with that normal control; and
3) diagnosing the selected subject of step 2) with high risk of cancer or with cancer analysis of step 2).

### ADVANTAGEOUS EFFECT

As explained hereinbefore, the present invention provides a method to distinguish the cancer patient group from the normal group efficiently by performing sequence analysis or quantitative analysis with marker glycoprotein isoforms having cancer specific glycochain that causes quantitative changes in various cancer cells. The method of the present invention facilitates fast diagnosis of cancer with the sample taken from a test subject based on the information about marker glycoprotein isoforms having cancer specific glycochain obtained from the quantitative analysis of the marker peptide generated from hydrolysis of a protein. Therefore, the selected peptide can be effectively used as a marker for cancer diagnosis.

### BRIEF DESCRIPTION OF THE DRAWINGS

The application of the preferred embodiments of the present invention is best understood with reference to the accompanying drawings, wherein:

FIG. 1 is a diagram illustrating the result of MRM with the marker peptide VFSLQWGEVK (SEQ ID NO:1) generated from the trypsin hydrolysis of complement factor I derived polypeptides in the lung cancer patient blood sample and the normal control blood sample.

FIG. 2 is a diagram illustrating the ROC (Receiver Operating Characteristic) curve obtained from the analysis with the target peptide VFSLQWGEVK (SEQ ID NO:1) to investigate the difference between the lung cancer blood sample and the control blood sample.

FIG. 3 is a diagram illustrating the result of CFI markers at the protein level by ELISA analysis for lung cancer patient blood samples (small-cell lung cancer; LG-SC, adenocarcinoma lung cancer; LC-AD, squamous-cell ung cancer; SQ-LC) and control blood samples.

### DESCRIPTION OF THE PREFERRED EMBODIMENTS

Hereinafter, the present invention is described in detail.

The present invention provides a method for diagnosing cancer by using the information on specific glycosylation of a glycoprotein.

Particularly, the present invention provides a method for diagnosing cancer by using one or more peptides selected from the following steps: separating glycoproteins containing cancer specific glycochain structure from the sample by using lectin; obtaining peptides by hydrolyzing the separated glycoproteins; selecting marker peptides able to detect quantitative changes in cancer specific glycosylated glycoproteins among the peptide samples obtained from the above hydrolysis; and diagnosing cancer by using one or more marker peptides selected above.

In a preferred embodiment of the present invention, the method composed of the following steps is preferably provided to give information useful for diagnosing cancer, but not always limited thereto:

The present invention provides a method for diagnosing cancer comprising the following steps:

1) separating and concentrating glycoproteins from the sample obtained from a test subject;

2) preparing polypeptides by hydrolyzing the glycoproteins of step 1;

3) sequencing and/or quantitative-analyzing the polypeptides of step 2); and

4) diagnosing the test subject with high risk of cancer or with cancer if the polypeptide composed of the amino acid sequence represented by SEQ ID NO:1 and/or the polypeptide having the molecular weight of 1191.6 is detected from the sequencing or quantitative analysis of step 3).

In the above method, the separation and concentration of glycoproteins in step 1 are preferably performed by treating the sample taken from the test subject with lectin.

In the above method, the sample of step 1) is the one obtainable from living things having proteins containing information related to cancer development and progress, which is exemplified by living tissue, cell line established from culture of the living tissue or the culture fluid thereof, saliva, and blood, etc. Particularly, those glycoproteins which contain the information about cancer are secreted outside of the cells into media or shed out of the cell membrane into the media once they finish their jobs. Therefore, culturing media for the culture of various cancer cell lines and blood samples of patients can be good experimental samples useful for the detection of cancer markers such as glycoproteins containing information about cancer. In the case of blood sample, there is a huge difference in concentrations of proteins composing blood. Therefore, the complexity of the sample can be minimized by the pre-treatment with high concentration protein elimination column [for example MARS (Multiple Affinity Removal System)]. However, such pre-treatment process to eliminate high concentration proteins is preferably omitted if possible unless there are problems of sensitivity and reproducibility of target markers.

The present invention also provides a method for diagnosing cancer comprising the following steps:

1) separating and concentrating glycoproteins from the sample obtained from a test subject;

2) preparing polypeptides by hydrolyzing the glycoproteins of step 1;

3) sequencing and/or quantitative-analyzing the polypeptides of step 2); and

4) diagnosing the test subject with high risk of cancer or with cancer if the polypeptide composed of the amino acid sequence selected from the group consisting of the sequences represented by SEQ ID NO:1 through SEQ ID NO:19, or the polypeptide having the molecular weight of 1191.6, 993.4, 979.6, 1113.6, 1296.7, 1093.7, 1014.6, 1195.6, 1370.8, 1369.7, 1291.7, 1354.8, 1140.6, 1026.5, 1250.6, 1115.6, 1139.6, 1097.6, or 1183.6 is detected from the sequencing or quantitative analysis of step 3).

The quantitative analysis in this method is preferably performed with the polypeptides obtained from hydrolysis of one or more marker glycoproteins selected from the group consisting of complement factor I, alpha-1-acid glycoprotein, haptoglobin, complement C4, lumican (LMC), alpha-1-antichymotrypsin (SERPINA3), alpha-1-antitrypsin (SERPINA1), alpha-2-HS-glycoprotein (AHSG), ceruloplasmin (CP), complement C3, fibronectin (FN1), galectin-3-binding protein (LGALS3BP), hemopexin (HPX), kallistatin (SERPINA4), kininogen-1 (KNG1), plasma protease C1 inhibitor (SERPING1), plasminogen (LPA), selenoprotein P (SEPP1), and serum paraoxonase/arylesterase 1 (PON1), but not always limited thereto.

In this method, the separation and concentration of a glycoprotein in step 1 are preferably performed after treating the sample obtained from a test subject with lectin.

In this method, the ‘cancer specific glycochain’ of a glycoprotein related to the occurrence of cancer indicates the aberrant glycosylation happening in cancer patients or in those having history of cancer, unlike in normal people. Such aberrant glycosylation is observed in those glycochains that are linked to asparagine, threonin, or serine residue. Such cancer specific glycochain shares a glycosylation site with a normal glycochain with presenting glycan microheterogeneity there. Therefore, a cancer specific glycochain is a part of many glycan-isoforms existing in a glycosylation site, which exists nonstoichiometrically in a very small portion by the total volume of a protein. To measure the quantitative changes in the cancer specific glycochain with a certainty, it is preferred to separate and concentrate the specific glycochains from various other glycan-isoforms, but not always limited thereto.

In this method, lectin can be used to separate and concentrate the isoform having a specific glycochain of interest from various glycan-isoforms demonstrating different glycochain structures. This method is taking advantage of the selectivity of lectin to the glycostructure, suggesting that it facilitates selective separation and concentration of marker glycoproteins having specific glycochain structures. According to the structure of glycochain wanted to be separated and concentrated, a variety of lectins such as ConA, WGA, Jacalin, SNA, AAL, L-PHA, PNA, LCA, ABA, DBA, DSA, ECA, SBA, SSA, UEA, VVL, or BPL can be used singly or as a combination. To separate proteins having different glycan-isoforms selectively from the total sample, various lectins can be used.

In this method, in order to investigate the quantitative changes of fucosylation presumed to increase in diverse cancer cells and cancer patient blood, glyco-isoform containing the glycochain having fucose structure was isolated and concentrated by using Aleuria aurantia lectin (AAL). At this time, the separation process of AAL selective glycoproteins naturally eliminates those proteins which do not have affinity to AAL. So, the complexity of a test sample can be much simplified without any additional pre-treatment process using MARS, etc.

In this method, it is preferred for the high molecular weight proteins separated by using lectin to be hydrolyzed into smaller molecular weight peptides in order to increase analysis efficiency. The process of preparing peptides by hydrolyzing glycoproteins can be performed by any biological method using various hydrolases or a chemical method using chemical reagents to induce hydrolysis in a specific amino acid site. The hydrolase herein can be one or more enzymes preferably selected from the group consisting of Arg-C, Asp-N, Glu-C, Lys-C, chymotrypsin, and trypsin, and among these trypsin is more preferred, but not always limited thereto. In this invention, when trypsin is used, one of those polypeptides listed in Table 1 can be a promising target among many concentrated polypeptides generated from glycoproteins of Table 1 including the Complementary factor I concentrated by lectin. However, it is clearly understood by those in the art that, in addition to the selected representative polypeptides of Table 1, other polypeptides having different sequences that can be separated from glycoproteins of Table 1 by hydrolysis can also be used for quantitative analysis of glycoprotein herein.

When other hydrolases than trypsin are used, for example Arg-C, Asp-N, Glu-C, Lys-C, or chymotrypsin, the peptides having different sequences but generated from the same glycoprotein can also be considered as target peptides. To increase hydrolysis efficiency or analysis efficiency of the generated peptides, the pre-treatment processes generally known as denaturation, reduction, and cysteine alkylation can be performed before hydrolysis. Therefore, the peptides containing cysteine its molecular weight can been changed by the pre-treatment or methionine its molecular weight can been changed by oxidation can also be considered as targets.

In this method, quantitative analysis of the hydrolyzed peptides obtained from both the normal group and the patient group enables the detection of quantitative changes of aberrantly glycosylated marker glycoproteins in relation to cancer development. Particularly, culturing media for various cancer cell lines and patient blood samples are good test samples to extract glycoproteins containing information about cancer, which are cancer markers. Herein, the said cancer includes every kind of cancer that can induce cancer specific aberrant glycosylation, which is exemplified by liver cancer, colon cancer, stomach cancer, lung cancer, uterine cancer, breast cancer, prostate cancer, thyroid cancer, and pancreatic cancer, and among them lung cancer is more preferred.

In this invention, the present inventors performed quantitative analysis of the marker polypeptide candidates as representatives of glycoproteins in order to examine the quantitative changes in fucosylated glycoproteins particularly increased in lung cancer patient blood, by which the present inventors completed this invention with providing a method for diagnosing cancer using the said peptide markers (see Table 1).

In the above method, the marker peptide selected from the peptide sample obtained by hydrolysis and concentration by using lectin can be one or more peptides originated from one glycoprotein or originated from different glycoproteins (see Table 1). Therefore, two or more marker peptides can be used for the sample analysis.

In this invention, the method used for quantitative analysis of the hydrolyzed peptides including maker peptides can be immuno-precipitation/immuno-blotting using an antibody specific to the peptide or mass spectrometry. In particular, mass spectrometry has no worry about obtaining the antibody against the target peptide, which means there is no limit in selecting target peptides. In addition, high speed/high sensitive analysis is another advantage of this mass spectrometry. As the mass spectrometry, multiple reaction monitoring (MRM) performing quantification by adding a stable isotope standard material labeled with an isotope into a sample as an internal standard material, or the method performing quantification by labeling a target peptide with a marker labeled with an isotope (iTRAQ, ICAT etc.) can be used.

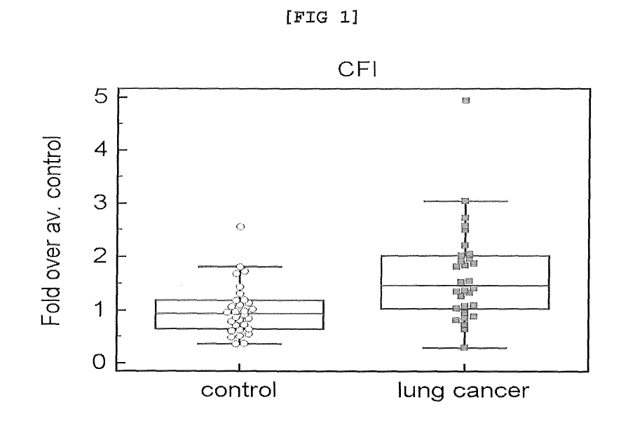

In an example of the present invention, quantitative analysis was performed according to the method of the invention with the peptide markers listed in Table 1 obtained from blood samples of lung cancer patients and of those with no opinion of cancer. FIG. 1 shows the result of multiple reaction monitoring (MRM) mass spectrometry with the marker peptide VFSLQWGEVK (SEQ ID NO:1) originated from complement factor I of Table 1, in which 30 blood samples obtained from clinically diagnosed lung cancer patients and 30 control blood samples from normal people of no opinion of cancer were used. The analysis was performed in triplicate. The values of total 60 samples analyzed were normalized by the mean value obtained from comparing them with the control blood samples (30), which were presented in box-and-whisker plot. The mean value of the marker peptides obtained from 30 lung cancer patients was 1.66 fold higher than that of the 30 control blood samples (see Table 1 and FIG. 1).

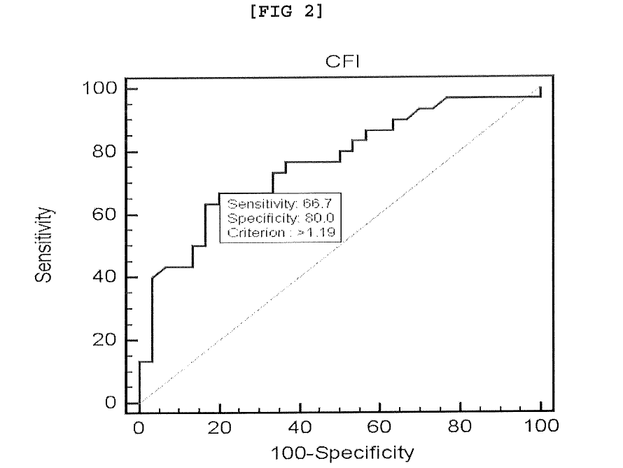

FIG. 2 presents the ROC (Receiver Operating Characteristic) curve illustrating the difference between 30 lung cancer blood samples and 30 control blood samples used in FIG. 1. AUROC (area under ROC) was 0.762, which means 80.0% marker specificity and 66.7% sensitivity. From the above result, it was confirmed that the analysis with blood samples using the marker peptide VFSLQWGEVK (SEQ ID NO:1) originated from complement factor I of the present invention can be efficient in identifying lung cancer patients from normal people (see FIG. 2).

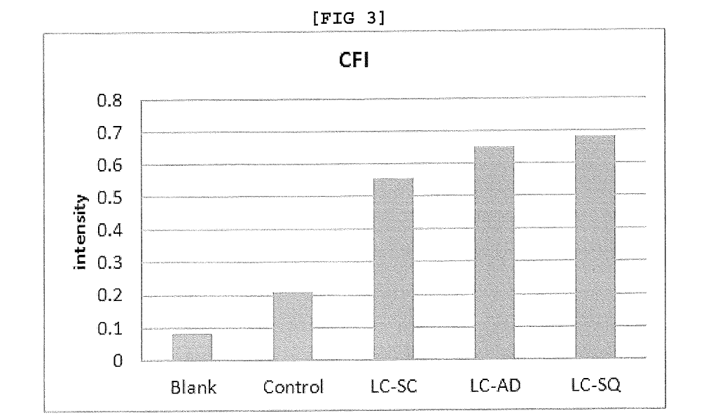

FIG. 3 presents to investigate Complement factor I can be used as a lung cancer marker, CFI markers of the present invention was analysed at the protein level by ELISA. The result of CFI level for lung cancer patient blood samples (small-cell lung cancer; LG-SC, adenocarcinoma lung cancer; LC-AD, squamous-cell ung cancer; SQ-LC) were significantly increased compared with control blood samples. The result of ELISA analysis same as the result of the present invention of quantitative analysis which is used polypeptide marker in <Example 3>. So that it can be effectively used as a lung cancer marker (see FIG. 3).

In this method using the peptide marker of the present invention, reliability of the marker glycoprotein developed herein can be more increased when the quantitative analysis is performed with the combination of one or more other marker peptides that can be generated by hydrolysis of the same marker glycoprotein. Further, the inventors performed quantitative analysis by MRM of other polypeptides generated from other glycoproteins such as alpha-1-acid glycoprotein, haptoglobin, complement C4, lumican (LUM), alpha-1-antichymotrypsin (SERPINA3), alpha-1-antitrypsin (SERPINA1), alpha-2-HS-glycoprotein (AHSG), ceruloplasmin (CP), complement C3 (C3), fibronectin (FN1), galectin-3-binding protein (LGALS3BP), hemopexin (HPX), kallistatin (SERPINA4), kininogen-1 (KNG1), plasma protease C1 inhibitor (SERPING1), plasminogen (LPA), selenoprotein P (SEPP1), and serum paraoxonase/arylesterase 1 (PON1) obtained from blood samples during the preparation of marker peptides from complement factor I (see Table 1). As a result, as shown in Table 2, those polypeptides originated from the said glycoproteins, analyzed together with the said marker peptide, were confirmed to be the marker peptides that can distinguish lung cancer patient samples from control samples (see Table 2).

Therefore, it was confirmed that the marker peptides developed in this invention can be effectively used to distinguish lung cancer patients from normal people via blood analysis. It has also been confirmed in this invention that the reliability of the marker glycoprotein of the present invention can be increased when the quantitative analysis is performed with the combination of one or more marker peptides of the invention generated by hydrolysis of the same marker glycoprotein or the combination of one or more marker peptides of the invention generated by hydrolysis of different glycoproteins demonstrating aberrant glycosylation over cancer progress.

The present invention also provides an antibody specifically binding to the polypeptide composed of the amino acid sequence represented by SEQ ID NO:1 or a kit for diagnosing cancer comprising the same.

The present invention also provides a kit for diagnosing cancer comprising an antibody specifically binding to each polypeptide composed of the amino acid sequence respectively represented by SEQ ID NO:1 through SEQ ID NO:19 or a combination of those antibodies.

In this invention, the cancer is liver cancer, colon cancer, stomach cancer, lung cancer, uterine cancer, breast cancer, prostate cancer, thyroid cancer, and pancreatic cancer, and among them lung cancer is more preferred, but not always limited thereto.

The said kit is to detect quantitative changes in marker peptides generated by hydrolysis of the test sample, indicating that the kit enables the screening, diagnosing, or monitoring cancer by sorting out the test subject with cancer.

The said polypeptides or peptides labeled with each isotope can be added in the said kit as standard materials.

The antibody used in the kit includes a polyclonal antibody, a monoclonal antibody, or a fragment binding to epitope. The polyclonal antibody herein can be produced by the conventional method comprising the following steps: injecting one of the said peptide markers to an animal, and obtaining serum containing a target antibody from the blood taken from the animal. The polyclonal antibody can be purified by any purification method known to those in the art and can be produced from any random host such as goat, rabbit, sheep, monkey, horse, pig, cow, and dog. The monoclonal antibody herein can be produced by any technique to provide an antibody molecule via continuous culture of a cell line. The said technique is exemplified by hybridoma technique, human-B-cell hybridoma technique, and EBV-hybridoma technique, but not always limited thereto (Kohler G et al., Nature 256:495-497, 1975; Kozbor D et al., J Immunol Methods 81:31-42, 1985; Cote R J et al., Proc Natl Acad Sci 80:2026-2030, 1983; and Cole S P et al., Mol Cell Biol 62:109-120, 1984). An antibody fragment containing a specific binding region to one of the said peptide markers can be prepared (Huse W D et al., Science 254: 1275-1281, 1989). The method for preparing such antibody against the peptide having a specific sequence is well known to those in the art.

The antibody included in the kit can be conjugated to the solid substrate in order to make the washing, separation of a complex, or other procedures thereafter, easy. The solid substrate herein is exemplified by synthetic resin, nitrocellulose, glass plate, metal plate, glass fiber, microsphere, and microbead, but not always limited thereto. The synthetic resin herein is exemplified by polyester, polyvinyl chloride, polystyrene, polypropylene, PVDF, and nylon, but not always limited thereto.

In the kit, the sample can be diluted in advance properly to contact the sample obtained from the test subject with the antibody binding specifically to one or those peptide markers conjugated to the solid substrate.

The kit is also advantageous in selecting marker peptides by eliminating additional proteins non-conjugated with the antibody by washing after contacting the sample obtained from the test subject with the antibody binding specifically to one of those peptide markers attached on the solid substrate.

The kit can additionally include the antibody for detection that is specifically binding to the said peptide marker. The antibody for detection can be a conjugate labeled with coloring enzyme, fluorescent material, isotope, or colloid, and more preferably the secondary antibody capable of binding specifically to the said marker, but not always limited thereto. The coloring enzyme herein can be peroxidase, alkaline phosphatase, or acid phosphatase (for example: horseradish peroxidase), but not always limited thereto. The fluorescent material herein can be fluorescein carboxylic acid (FCA), fluorescein Isothiocyanate (FITC), fluorescein thiourea (FTH), 7-acetoxycoumarin-3-yl, fluorescein-5-yl, fluorescein-6-yl, 2′,7′-dichloro fluorescein-5-yl, 2′,7′-dichloro fluorescein-6-yl, dihydrotetramethylrhodamine-4-yl, tetramethylrhodamine-5-yl, tetramethylrhodamine-6-yl, 4,4-difluoro-5,7-dimethyl-4-bora-3a,4a-diaza-s-indacene-3-ethyl, or 4,4-difluoro-5,7-diphenyl-4-bora-3a, but not always limited thereto.

In this invention, the kit can additionally include a washing buffer or an eluent in order to keep the conjugated peptide marker alone and to eliminate non-conjugated proteins, enzymes, and substrates.

The present invention also provides a biochip for diagnosing cancer wherein biomolecules specifically binding to the polypeptide composed of the amino acid sequence represented by SEQ ID NO:1 are integrated on the solid substrate.

In addition, the present invention provides a biochip for diagnosing cancer which is characterized by the further integration of biomolecules specifically binding to each polypeptide composed of the amino acid sequence represented by SEQ ID NO:1 through SEQ ID NO:19.

The polypeptide originated from the selected glycoprotein is preferably the polypeptide labeled with a stable isotope, but not always limited thereto.

The said biochip facilitates the monitoring, diagnosing, and screening of cancer by detecting quantitative changes in the marker peptides obtained from the sample of the test subject through hydrolysis, leading to the diagnosing the subject with cancer or not.

The said biomolecule is preferably an antibody or an aptamer, but not always limited thereto. The biomolecule indicates not only the small molecule such as primary metabolites, secondary metabolites, and natural substances but also the organic molecule produced by a living organism including the macromolecule such as proteins, polysaccharides, and nucleic acids. The aptamer herein is an oligonucleotide or a peptide binding to a specific target molecule.

The solid substrate herein is preferably selected from the group consisting of plastic, glass, metal, and silicone, but not always limited thereto.

The cancer herein is preferably selected from the group consisting of liver cancer, colon cancer, stomach cancer, lung cancer, uterine cancer, breast cancer, prostate cancer, thyroid cancer, and pancreatic cancer, and among them lung cancer is more preferred, but not always limited thereto.

The present invention also provides a method for diagnosing cancer comprising the following steps:
1) measuring the expression level of Complement factor I from the sample obtained from a test subject;
2) selecting a subject demonstrating the increased expression level of CFI, compared with that normal control; and
3) diagnosing the selected subject of step 2) with high risk of cancer or with cancer analysis of step 2).

The cancer herein is preferably selected from the group consisting of liver cancer, colon cancer, stomach cancer, lung cancer, uterine cancer, breast cancer, prostate cancer, thyroid cancer, and pancreatic cancer, and among them lung cancer is more preferred, but not always limited thereto.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### EXAMPLES

### Example 1

### Sample Preparation

Thirty (30) human plasma samples were obtained from clinically diagnosed lung cancer patients and another 30 human plasma samples were obtained from the control people confirmed not to have cancer. AAL-lectin specific protein sample was separated from the plasma (equal amount for each) by using AAL (aleuria aurantia lectin) lectin demonstrating specific affinity to the glycoprotein having fucose glycochain. The separated sample was hydrolyzed to obtain the polypeptide sample for each plasma sample. As a support to fix lectin, various supports including agarose beads and magnetic beads can be used. In the analysis of clinical blood samples, strepavidine-magnetic beads were used to fix lectin. Particularly, the blood samples of the lung cancer group and the normal control group were treated with AAL-biotin-strepavidine-magnetic beads in phosphate-buffered saline (PBS), which stood at 4° C. for 12 hours. Lectin conjugated proteins were washed with PBS three times and the proteins were separated by using 2 M urea/DDT solution. The obtained protein was treated with iodoacetamide (IAA), which was diluted with 50 mM ammonium bicarbonate. The protein was then hydrolyzed by using trypsin at 37° C. overnight. The hydrolyzed peptide was dried under reduced pressure.

### Example 2

### Marker Polypeptide Sequencing by Peptide Analysis

HPLC (high-performance liquid chromatography) was performed to analyze the samples prepared in <Example 1>.

Particularly, LC/ESI-MS/MS with electrospray ionization (ESI) mass spectrometer connecting HPLC (high-performance liquid chromatography) was performed (trap column, C18, 5 um, 300×5 mm, and analytical column, C18, 5 um, 75 um×10 cm). The sample proteome concentrated by lectin, prepared by the same manner as described in <Example 1>, was hydrolyzed by using trypsin, and some of the peptide samples were 10-fold diluted, which was loaded in liquid chromatography attached with mass spectrometer by 10 μl for the analysis. Based on the result of the mass spectrometry, search engine such as MASCOT and SEQUEST was used to confirm the polypeptides obtained by hydrolysis of the target glycoproteins concentrated by AAL-lectin. The hydrolyzed polypeptide derived from the target glycoprotein was designated as a representative, followed by MRM MS for the quantification to confirm whether or not the target glycoprotein could be used as a cancer marker.

The below [Table 1] presents the representative target polypeptides selected among those peptides generated by hydrolyzing the selected target glycoproteins by using trypsin. It is also general idea accepted by those in the art that those peptides generated from the same glycoprotein but by using other hydrolases than trypsin, for example Arg-C, Asp-N, Glu-C, Lys-C, and chymotrypsin, etc, can also be members of the target marker polypeptide panel.

TABLE 1

| Sequence | | Marker | Peptide |
|---|---|---|---|
| Number | Marker Protein | Peptide | Mass |
| 1 | Complement factor I | VFSLQWGEVK | 1191.6 |
| 2 | Alpha-1- | TEDTIFLR | 993.4 |
| | acidglycoprotein | | |
| 3 | Haptoglobin | VGYVSGWGR | 979.6 |
| 4 | Complement C4-A | VGDTLNLNLR | 1113.6 |
| 5 | Lumican | SLEDLQLTHNK | 1296.7 |
| 6 | Alpha-1-anti- | NLAVSQWHK | 1093.7 |
| | chymotrypsin | | |
| 7 | Alpha-1-antitrypsin | SVLGQLGITK | 1014.6 |
| 8 | Alpha-2-HS- | HTLNQIDEVK | 1195.6 |
| | glycoprotein | | |
| 9 | Ceruloplasmin | GAYPLSIEPIGVR | 1370.8 |
| 10 | Complement C3 | TIYTPGSTVLYR | 1369.7 |
| 11 | Fibronectin | DLQFVEVTDVK | 1291.7 |
| 12 | Galectin-3- | SDLAVPSELALLK | 1354.8 |
| | bindingprotein | | |
| 13 | Hemopexin | GGYTLVSGYPK | 1140.6 |
| 14 | Kallistatin | LGFTDLFSK | 1026.5 |
| 15 | Kininogen-1 | TVGSDTFYSFK | 1250.6 |
| 16 | Plasma protease C1 | LLDSLPSDTR | 1115.6 |
| | inhibitor | | |
| 17 | Plasminogen | EAQLPVIENK | 1139.6 |
| 18 | Selenoprotein P | LPTDSELAPR | 1097.6 |
| 19 | Serum paraoxonase/ | IQNILTEEPK | 1183.6 |
| | arylesterase 1 | | |

As a result, as shown in Table 1, the combination of two or more peptides which can be produced from the same glycoprotein via hydrolysis, in addition to the marker peptide VFSLQWGEVK (SEQ ID NO:1) originated from complement factor I, can also be used as the representative of the marker glycoprotein complement factor I. The marker peptide originated from complement factor I was confirmed to be effectively used for diagnosing cancer by being paired with other polypeptides originated from different target glycoproteins existing in the same sample.

### Example 3

### Quantitative Analysis of Marker Polypeptides Using Mass Spectrometry

To analyze the samples prepared in <Example 1>, isotope-labeled standard materials having the same sequence as the marker polypeptide of [Table 1] were prepared. The prepared standard materials were added to each peptide sample prepared in <Example 1> at the same concentration, suggesting that they became internal standard materials for the quantitative analysis. Then, LC/MRM quantitative mass spectrometry was performed in triplicate for each sample. In the case that the target polypeptide existed only at a very low concentration in the blood sample or in the case that the analysis of the target polypeptide is interrupted by other peptides existing in the same sample, the target marker peptide cannot be detected from the peptide sample through MRM quantitative mass spectrometry. So, if that is the case, an anti-peptide antibody selective to the target marker peptide was used according to the conventional method informed to those in the art. That is, the target marker peptide was obtained and concentrated from the peptide sample prepared above, and then quantitative analysis was performed. At this time, the poly or monoclonal antibody was directly fixed on the polymeric solid substrate or the magnetic solid substrate or attached with an avidine-biotin linker for the convenience in experiment. The values obtained from the quantitative analysis with the marker peptide VFSLQWGEVK (SEQ ID NO:1) originated from complement factor I obtained from the total 60 human plasma samples were normalized by using the mean value of the 30 control samples, which were presented by box-and-whisker plot.

As a result, as shown in FIG. 1, the average level of the marker peptide in 30 lung cancer patient samples was 1.66 times higher than the average level of the marker peptide in 30 control samples (FIG. 1).

As shown in FIG. 2, the difference between 30 lung cancer patient plasma samples and 30 control plasma samples is presented as ROC (Receiver Operating Characteristic) curve by using the results of analysis performed with the target peptide VFSLQWGEVK (SEQ ID NO:1). Herein, AUROC is 0.762, P-value is <0.0001.

In addition, the below [Table 2] presents the results of additional MRM mass spectrometry performed with the marker polypeptides shown in [Table 1] generated from other blood glycoproteins such as alpha-1-acid glycoprotein, haptoglobin, complement C4, lumican (LMC), alpha-1-antichymotrypsin (SERPINA3), alpha-1-antitrypsin (SERPINA1), alpha-2-HS-glycoprotein (AHSG), ceruloplasmin (CP), complement C3 (C3), fibronectin (FN1), galectin-3-binding protein (LGALS3BP), hemopexin (HPX), kallistatin (SERPINA4), kininogen-1 (KNG1), plasma protease C1 inhibitor (SERPING1), plasminogen (LPA), selenoprotein P (SEPP1), and serum paraoxonase/arylesterase 1 (PON1), while the polypeptide originated from complement factor I was generated.

TABLE 2

| | | | av. AD/ | ROC |
|---|---|---|---|---|
| Sequence | | | av. | Curve |
| Number | Marker Protein | Marker Peptide | control | (AUC) |
| 1 | Complement factor I | VFSLQWGEVK | 1.66 | 0.762 |
| 2 | Alpha-1-acidglycoprotein | TEDTIFLR | 1.96 | 0.758 |
| 3 | Haptoglobin | VGYVSGWGR | 2.16 | 0.678 |
| 4 | Complement C4-A | VGDTLNLNLR | 1.33 | 0.673 |
| 5 | Lumican | SLEDLQLTHNK | 1.80 | 0.841 |
| 6 | Alpha-1-antichymotrypsin | NLAVSQWHK | 2.62 | 0.847 |
| 7 | Alpha-1-antitrypsin | SVLGQLGITK | 1.77 | 0.792 |
| 8 | Alpha-2-HS-glycoprotein | HTLNQIDEVK | 1.51 | 0.767 |
| 9 | Ceruloplasmin | GAYPLSIEPIGVR | 1.99 | 0.847 |
| 10 | Complement C3 | TIYTPGSTVLYR | 1.75 | 0.821 |
| 11 | Fibronectin | DLQFVEVTDVK | 4.28 | 0.929 |
| 12 | Galectin-3-bindingprotein | SDLAVPSELALLK | 2.19 | 0.856 |
| 13 | Hemopexin | GGYTLVSGYPK | 2.07 | 0.856 |
| 14 | Kallistatin | LGFTDLFSK | 2.01 | 0.840 |
| 15 | Kininogen-1 | TVGSDTFYSFK | 2.07 | 0.916 |
| 16 | Plasma protease C1 | LLDSLPSDTR | 2.40 | 0.911 |
| | inhibitor | | | |
| 17 | Plasminogen | EAQLPVIENK | 1.88 | 0.867 |
| 18 | Selenoprotein P | LPTDSELAPR | 1.39 | 0.805 |
| 19 | Serum | IQNILTEEPK | 2.38 | 0.890 |
| | paraoxonase/arylesterase 1 | | | |

As a result, as shown in Table 2, the marker target peptides showed difference between the lung cancer group and the control group. So, these marker target peptides generated from the said marker glycoproteins by hydrolysis can also be marker peptides for lung cancer just like the representative polypeptides originated from complement factor I. When these marker polypeptides are analyzed together with the marker polypeptide VFSLQWGEVK (SEQ ID NO:1) originated from complement factor I, more reliable information for the diagnosis of cancer can be obtained.

### Example 4

### ELISA was Performed to Analyze a Marker Protein

To investigate whether Complement factor I can be used as a lung cancer marker, CFI markers of the present invention were analysed at the protein level by ELISA.

Particularly, to determine the level of complement factor I from sera, the surface of plates was blocked with 200 ul of protein-free blocking buffer solution for 1 h at room temperature. Pooled serum samples, prepared by pooling each 10 cases of control and lung cancer groups, were diluted with PBS by 200 and 400 folds for analysis of complement factor I. Two hundred micoliters of diluted serum samples were added to each well and incubated at 37° C. for 2 h. After washing with 200 ul of PBS three times, primary antibody was added and allowed to incubate for 2 h. The unbound material was extensively washed with PBS. Secondary antibody labeled with horseradish peroxidase (HRP) was diluted to 1:2000 and allowed to bind for 30 min. The substrate solution was treated for 10-30 min and the colored reaction product was measured using an automated ELISA reader at 450 nm.

As shown in FIG. 3, the result of CFI level for lung cancer patient blood samples (small-cell lung cancer; LG-SC, adenocarcinoma lung cancer; LC-AD, squamous-cell ung cancer; SQ-LC) were significantly increased compared with control blood samples. The result of ELISA analysis was in agreement with the result of the present invention of quantitative analysis which is used polypeptide marker in <Example 3>. So that it can be effectively used as a lung cancer marker (see FIG. 3).

### INDUSTRIAL APPLICABILITY

As explained hereinbefore, the present invention provides marker peptides which are screened by the steps of: separating and concentrating glycoproteins aberrantly glycosylated according to the cancer development from lung cancer patient blood by using lectin; obtaining polypeptides by hydrolyzing the said glycoproteins; and selecting marker peptides hydrolyzed from the marker glycoprotein (complement factor I) demonstrating cancer specific glycosylation through sequence analysis and quantitative analysis. The marker peptides can be effectively used as cancer diagnosis markers and for the diagnosis of cancer.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for diagnosing cancer, said method comprising
1) separating and concentrating glycoproteins from the sample obtained from a test subject;
2) preparing polypeptides by hydrolyzing the glycoproteins of step 1);
3) sequencing and quantitative-analyzing the polypeptides of step 2); and
4) diagnosing the test subject with high risk of cancer or with cancer if the polypeptide composed of the amino acid sequence represented by SEQ ID NO:1 or the polypeptide having the molecular weight of 1191.6 is detected from the sequencing or quantitative analysis of step 3).

2. The method for diagnosing cancer according to claim 1, wherein the sample of step 1) is selected from the group consisting of blood, serum, and plasma.

3. The method for diagnosing cancer according to claim 1, wherein the separation and concentration of step 1) is performed by treating the sample isolated from the test subject with lectin.

4. The method for diagnosing cancer according to claim 3, wherein the lectin of step 1) is the one or a combination of at least two of those selected from the group consisting of ConA, WGA, Jacalin, SNA, AAL, L-PHA, PNA, LCA, ABA, DBA, DSA, ECA, SBA, SSA, UEA, VVL, and BPL.

5. The method for diagnosing cancer according to claim 1, wherein the hydrolysis in step 2) is performed with one of the enzymes selected from the group consisting of Arg-C, Asp-N, Glu-C, Lys-C, chymotrypsin, and trypsin.

6. The method for diagnosing cancer according to claim 1, further comprising an additional step of concentrating the polypeptide of step 2) using an antibody before the analysis of step 3).

7. The method for diagnosing cancer according to claim 1, wherein the quantitative analysis is performed with the polypeptide obtained by hydrolysis of the glycoprotein complement factor I.

8. The method for diagnosing cancer according to claim 1, further comprising the step of diagnosing the test subject with high risk of cancer or with cancer when the polypeptide composed of one of those amino acid sequences each represented by SEQ ID NO:2 through NO: 19 is confirmed by sequence analysis in step 4) or when the polypeptide having any of those molecular weights, 993.4, 979.6, 1113.6, 1296.7, 1093.7, 1014.6, 1195.6, 1370.8, 1369.7, 1291.7, 1354.8, 1140.6, 1026.5, 1250.6, 1115.6, 1139.6, 1097.6, and 1183.6 is confirmed by quantitative analysis in step 4).

9. The method for diagnosing cancer according to claim 8, wherein the quantitative analysis is performed with the polypeptide obtained from one or more marker glycoproteins via hydrolysis which are selected from the group consisting of alpha-1-acid glycoprotein, haptoglobin, complement C4, lumican (LMC), alpha-1-antichymotrypsin (SERPINA3), alpha-1-antitrypsin (SERPINA1), alpha-2-HS-glycoprotein (AHSG), ceruloplasmin (CP), complement C3 (C3), fibronectin (FN1), galectin-3-binding protein (LGALS3BP), hemopexin (HPX), kallistatin (SERPINA4), kininogen-1 (KNG1), plasma protease C1 inhibitor (SERPING1), plasminogen (LPA), selenoprotein P (SEPP1), and serum paraoxonase/arylesterase 1 (PON1).

10. The method for diagnosing cancer according to claim 8, wherein the sequence analysis confirms that the polypeptide separated from the glycoprotein complement factor I by hydrolysis is composed of the amino acid sequence represented by SEQ ID NO:1, the polypeptide separated from the alpha-1-acid glycoprotein by hydrolysis is composed of the amino acid sequence represented by SEQ ID NO:2, the polypeptide separated from the haptoglobin by hydrolysis is composed of the amino acid sequence represented by SEQ ID NO:3, the polypeptide separated from the complement C4 by hydrolysis is composed of the amino acid sequence represented by SEQ ID NO:4, the polypeptide separated from the lumican by hydrolysis is composed of the amino acid sequence represented by SEQ ID NO:5, the polypeptide separated from the alpha-1-antichymotrypsin by hydrolysis is composed of the amino acid sequence represented by SEQ ID NO:6, the polypeptide separated from the alpha-1-antitrypsin by hydrolysis is composed of the amino acid sequence represented by SEQ ID NO:7, the polypeptide separated from the alpha-2-HS-glycoprotein by hydrolysis is composed of the amino acid sequence represented by SEQ ID NO:8, the polypeptide separated from ceruloplasmin by hydrolysis is composed of the amino acid sequence represented by SEQ ID NO:9, the polypeptide separated from the complement C3 by hydrolysis is composed of the amino acid sequence represented by SEQ ID NO:10, the polypeptide separated from the fibronectin by hydrolysis is composed of the amino acid sequence represented by SEQ ID NO:11, the polypeptide separated from the galectin-3-binding protein by hydrolysis is composed of the amino acid sequence represented by SEQ ID NO:12, the polypeptide separated from the hemopexin by hydrolysis is composed of the amino acid sequence represented by SEQ ID NO:13, the polypeptide separated from the kallistatin by hydrolysis is composed of the amino acid sequence represented by SEQ ID NO:14, the polypeptide separated from the kininogen-1 by hydrolysis is composed of the amino acid sequence represented by SEQ ID NO:15, the polypeptide separated from the plasma protease C1 inhibitor by hydrolysis is composed of the amino acid sequence represented by SEQ ID NO:16, the polypeptide separated from the plasminogen by hydrolysis is composed of the amino acid sequence represented by SEQ ID NO:17, the polypeptide separated from the selenoprotein P by hydrolysis is composed of the amino acid sequence represented by SEQ ID NO:18, and the polypeptide separated from the serum paraoxonase/arylesterase 1 by hydrolysis is composed of the amino acid sequence represented by SEQ ID NO:19.

11. The method for diagnosing cancer according to claim 1, wherein the cancer is selected from the group consisting of liver cancer, colon cancer, stomach cancer, lung cancer, uterine cancer, breast cancer, prostate cancer, thyroid cancer, and pancreatic cancer.

12. A method for diagnosing cancer, said method comprising:
1) separating and concentrating glycoproteins from the sample obtained from a test subject;
2) preparing polypeptides by hydrolyzing the glycoproteins of step 1);
3) performing sequence analysis with the polypeptides of step 2); and
4) diagnosing the test subject with high risk of cancer or with cancer if the polypeptide composed of the amino acid sequence represented by SEQ ID NO:1 is detected from the sequence analysis of step 3).

13. The method for diagnosing cancer according to claim 12, further comprising the step of concentrating the polypeptide of step 2) by using an antibody before the analysis of step 3).

14. The method for diagnosing cancer according to claim 12, wherein the step of diagnosing the test subject with high risk of cancer or with cancer when the polypeptide composed of one of those amino acid sequences each represented by SEQ ID NO:2 through SEQ ID NO:19 is confirmed by sequence analysis in step 4).

15. The method for diagnosing cancer according to claim 12, wherein the separation and concentration of step 1) is performed by treating the sample isolated from the test subject with lectin.

16. A method for diagnosing cancer, said method comprising:
1) separating and concentrating glycoproteins from the sample obtained from a test subject;
2) preparing polypeptides by hydrolyzing the glycoproteins of step 1);
3) performing quantitative analysis with the polypeptides of step 2); and
4) diagnosing the test subject with high risk of cancer or with cancer if the polypeptide having the molecular weight of 1191.6 is detected from the quantitative analysis of step 3).

17. The method for diagnosing cancer according to claim 16, further comprising the step of concentrating the polypeptide of step 2) by using an antibody before the analysis of step 3).

18. The method for diagnosing cancer according to claim 16, further comprising the step of diagnosing the test subject with high risk of cancer or with cancer when the polypeptide having any of those molecular weights, 993.4, 979.6, 1113.6, 1296.7, 1093.7, 1014.6, 1195.6, 1370.8, 1369.7, 1291.7, 1354.8, 1140.6, 1026.5, 1250.6, 1115.6, 1139.6, 1097.6, and 1183.6 is confirmed by quantitative analysis in step 4).

19. A method for diagnosing cancer, said method comprising:
1) measuring the expression level of Complement factor I from the sample obtained from a test subject;
2) selecting a subject demonstrating the increased expression level of CFI, compared with that normal control; and
3) diagnosing the selected subject of step 2) with high risk of cancer or with cancer analysis of step 2).

20. The method for diagnosing cancer according to claim 19, wherein the cancer is selected from the group consisting of liver cancer, colon cancer, stomach cancer, lung cancer, uterine cancer, breast cancer, prostate cancer, thyroid cancer, and pancreatic cancer.


SEQUENCE LISTING (19 sequences)

SEQ ID NO: 1 - protein, 10 aa, Homo sapiens
VFSLQWGEVK

SEQ ID NO: 2 - protein, 8 aa, Homo sapiens
TEDTIFLR

SEQ ID NO: 3 - protein, 9 aa, Homo sapiens
VGYVSGWGR

SEQ ID NO: 4 - protein, 10 aa, Homo sapiens
VGDTLNLNLR

SEQ ID NO: 5 - protein, 11 aa, Homo sapiens
SLEDLQLTHNK

SEQ ID NO: 6 - protein, 10 aa, Homo sapiens
NLAVSQVVHK

SEQ ID NO: 7 - protein, 10 aa, Homo sapiens
SVLGQLGITK

SEQ ID NO: 8 - protein, 10 aa, Homo sapiens
HTLNQIDEVK

SEQ ID NO: 9 - protein, 13 aa, Homo sapiens
GAYPLSIEPIGVR

SEQ ID NO: 10 - protein, 12 aa, Homo sapiens
TIYTPGSTVLYR

SEQ ID NO: 11 - protein, 11 aa, Homo sapiens
DLQFVEVTDVK

SEQ ID NO: 12 - protein, 13 aa, Homo sapiens
SDLAVPSELALLK

SEQ ID NO: 13 - protein, 11 aa, Homo sapiens
GGYTLVSGYPK

SEQ ID NO: 14 - protein, 9 aa, Homo sapiens
LGFTDLFSK

SEQ ID NO: 15 - protein, 11 aa, Homo sapiens
TVGSDTFYSFK

SEQ ID NO: 16 - protein, 10 aa, Homo sapiens
LLDSLPSDTR

SEQ ID NO: 17 - protein, 10 aa, Homo sapiens
EAQLPVIENK

SEQ ID NO: 18 - protein, 10 aa, Homo sapiens
LPTDSELAPR

SEQ ID NO: 19 - protein, 10 aa, Homo sapiens
IQNILTEEPK